(19) United States
(12) Patent Application Publication
(10) Pub. No.: US 2022/0090690 A1
(43) Pub. Date: Mar. 24, 2022

(54) MULTI-WAY CONNECTOR

(71) Applicant: BIOCOMPATIBLES UK LIMITED, Surrey (GB)

(72) Inventors: Scott McGhee, Ottawa (CA); David Burnett, Camberley/Surrey (GB); Martina Marti, Basingstoke/Hampshire (GB); Joe Neale, Camberley/Surrey (GB); Jonathan Vince, Hampshire (GB)

(21) Appl. No.: 17/473,339

(22) Filed: Sep. 13, 2021

Related U.S. Application Data
(60) Provisional application No. 63/082,131, filed on Sep. 23, 2020.

(51) Int. Cl. F16K 11/085; F16K 31/60; F16K 27/06

(52) U.S. Cl. CPC F16K 11/0853; F16K 27/065; F16K 31/602

(57) ABSTRACT

One aspect of the present disclosure pertains to a connector for conveying a suspension of particles through a system, comprising: a housing (10) comprising an internal cavity (12) and a first passageway (14) for conveying fluid; two or more inlet conduits (16a, 16b, 16c, 16d) formed in the housing and arranged radially with respect to a longitudinal axis of the first passageway; and a rotatable component (18) received in the internal cavity of the housing and rotatable about the longitudinal axis of the first passageway such that a continuous flow path can be established between the first passageway and a selected inlet conduit; wherein an angle between each of the inlet conduits and the first passageway (22) is greater than 90 degrees. Other aspects pertain to systems that employ such a connector and to methods of using such systems.

### PRIORITY

The present application is a non-provisional of, and claims the benefit of priority under 35 U.S.C. § 119 to, U.S. Provisional Application Ser. No. 63/082,131, filed Sep. 23, 2020, the disclosure of which is herein incorporated herein by reference in its entirety for all purposes.

### FIELD

This disclosure relates to a connector for conveying a suspension of particles through a system, a system for conveying a suspension of particles and a method of conveying a suspension of particles through a system.

### BACKGROUND

In some systems, such as therapeutic systems for the treatment of disease, a fluid or suspension of particles is conveyed or delivered to a target, such as a cancerous tumour, through a conduit that includes a coupling. Where the fluid is a suspension of particles, such as radioactive microparticles, the microparticles can become trapped in the coupling. For example, some microparticles can become trapped in gaps that result from mechanically mismatched components in the coupling and on other occasions microparticles get trapped in regions of stagnant fluid along the conduit or coupling. As a consequence, these microparticles do not get successfully delivered to the target. One method of overcoming this problem is to convey the fluid at higher pressures or at higher flow rates however this increases the likelihood of leakages from the system.

In order to achieve effective treatment in a therapeutic system, substantially all the microparticles introduced are preferably delivered to the target. Failure to do so reduces the effectiveness of the treatment as a lower than required dose is delivered to the target due to the proportion of microparticles becoming trapped in the system. Where the microparticles are radioactive, microparticles leakage or failure to deliver the radioactive microparticles to the target is a particular problem because this results in contamination with radioactive material. Furthermore, as noted above, where the radioactive microparticles are being administered for the treatment of a condition it is desirable that all the intended microparticles are administered to the subject to ensure the correct dose is delivered for treating the condition.

In some therapeutic procedures it may be desirable to administer more than one source of fluid into the system. For the delivery of radioactive microparticles the microparticles are typically provided in vials containing a pre-determined dose per vial and so it may be necessary to administer more than one vial to achieve the required dose in the subject. Typically, this is achieved by disconnecting the first vial once its contents have been administered and then connecting to the next and each subsequent vial until the required dose has been administered. This is however time consuming, it may yield unacceptable radiation exposure to the operator, and there is still the risk that not all the contents of the vials will be administered to the target due to loses when connecting and disconnecting. In other procedures it may, at times, be desirable to administer non-therapeutic fluids in addition to the fluid containing therapeutic agent, for example, without having to connect and/or disconnect system components. For example, it may be desirable to administer contrast agent in solution (for example, Lipiodol®) in between administering doses of radioactive microparticles to assist with the visualisation of the procedure.

These and other challenges may be addressed by the present disclosure.

### SUMMARY

Connector

According to an aspect of the disclosure, there is provided a connector for conveying a suspension of particles through a system, said connector comprising: a housing comprising an internal cavity that comprises a first passageway having a longitudinal axis and first and second ends for conveying fluid and wherein the first passageway terminates in an outlet at the second end, two or more inlet conduits formed in the housing and arranged radially with respect to the longitudinal axis of the first passageway, and a rotatable component received in a portion of the internal cavity of the housing that is adjacent to the first passageway, the rotatable component being rotatable about the longitudinal axis of the first passageway, wherein the rotatable component comprises an internal second passageway having first and second ends, the first end of the second passageway in fluid communication with the first end of the first passageway, and wherein the rotatable component is rotatable such that the second end of the second passageway is selectively alignable with any one of the inlet conduits such that a continuous flow path is established between the first passageway and the selected inlet conduit.

Angle Relative to the First Passageway

In various embodiments, an angle between a longitudinal axis of each of the inlet conduits and the longitudinal axis of the first passageway of the housing is greater than 90 degrees. Where the angle between each of the inlet conduits and the first passageway is greater than 90 degrees, improved clearance of particles through the connector is ensured as regions of stagnant or recirculating fluid along the flow path are reduced. Moreover, the larger the angle between any given inlet conduit and the first passageway, the less fluid turbulence that exists as the fluid flows around any bend that is required within the rotatable component. Where the first passageway is coaxial with the rotational axis of the rotatable component the angle between each of the inlet conduits and the first passageway is generally the same for each inlet conduit. This allows the second passageway to be selectably alignable with each of the inlet conduits such that a continuous flow path is formed between the second passageway and each inlet conduit. The continuous flow path is preferably created to minimise disruptions in the flow path caused by mechanically mismatched parts where the angles between each of the inlet conduits and the first passageway are slightly different.

The angle of greater than 90 degrees between the selected inlet conduit and the first passageway is accommodated by a bend formed in the second passageway. The rotatable component is rotatable such that the second end of the second passageway is selectably alignable with any one of the inlet conduits and the first end of the second passageway is aligned with the first passageway. In various embodiments, a longitudinal axis of the second end of the second passageway is coaxial with a longitudinal axis of each of the inlet conduits and longitudinal axis of the first end of the second passageway is coaxial with the longitudinal axis of the first passageway, and the bend formed in the second passageway has an angle that is the same as the angle between the longitudinal axis of each of the inlet conduits and the longitudinal axis of the first passageway. As a result, the angle of greater than 90 degrees between the selected inlet conduit and the first passageway need not be formed at the junction between two different component parts. This reduces the problem associated with particles becoming trapped in gaps resulting from mechanically mismatched components. The junctions between different component parts instead exist at points where the resulting fluid path formed at that point is in a straight line, i.e. from the selected inlet conduit to the second end of the second passageway and from the first end of the second passageway to the first passageway. Even when neighbouring component parts are mechanically well matched, a small amount of fluid turbulence will be created at the point where they join and bends in the flow path also create fluid turbulence. By having the bend in the flow path within a single component and junctions between different component parts at different points in the connector means that fluid turbulence is minimised.

As noted above, in various embodiments, the angle between each of the inlet conduits and the first passageway is greater than 90 degrees, and the angle of the bend within the second passageway is preferably greater than 90 degrees. Preferably the angle between each of the inlet conduits and the first passageway is greater than 110 degrees, and the angle of the bend within the second passageway is preferably greater than 110 degrees. The angle between any given inlet conduit and the rotational axis of the rotatable component is measured from the part of the rotational axis of the rotatable component which extends through the housing but not including the first passageway. Nevertheless, the rotational axis of the rotatable component will in most cases be coaxial with the longitudinal axis of the first passageway. This is the preferred embodiment since with this embodiment the only bend in the flow path through the connector will be formed in the second passageway and this minimises fluid turbulence.

More typically the angle between each of the inlet conduits and the first passageway is greater than 120 degrees, for example, ranging from 120 degrees to 130 degrees to 140 degrees to 150 degrees to 160 degrees, or more. The larger the angle between the any given inlet conduit and the first passageway the less fluid turbulence will occur as the fluid flows around the bend in the second passageway. However as the connector comprises two or more inlet conduits, the two or more inlet conduits are offset radially from each other with respect to the longitudinal axis of the first passageway and so the angle between each inlet conduit and the first passageway cannot be as much as 180 degrees. There is therefore a balance between making the angle between each of the inlet conduits and the first passageway sufficiently large to reduce fluid turbulence through the bend but at the same time having the inlet conduits sufficiently offset radially from the longitudinal axis of the first passageway such that the inlet conduits are not crowded too close together.

The inlet conduits are ultimately connectable to feed lines for conveying fluid to the connecter and they need to be sufficiently splayed to make it easy for the operator to connect and disconnect feed lines easily and the operator should also be able to rotate the rotatable component and typically this is achieved by rotating a top part of the rotatable component which protrudes from the housing. The present inventors have identified that in order to achieve this balance, the angle between each of the inlet conduits and the first passageway is preferably greater than 120 degrees and less than 140 degrees.

Housing/First Passageway

The housing comprises an internal cavity, a first portion of which includes a first passageway arranged about a longitudinal axis for conveying fluid and the inlet conduits are also formed in the housing. The housing preferably comprises a main body portion and the inlet conduits. In some embodiments, the inlet conduits, the internal cavity and the main body portion are integrally formed in a single piece.

The main body portion, the internal cavity and the inlet conduits may be formed from any material that is suitable for conveying fluids, in particular suspensions of particles. Preferably the material or materials which make up the main body portion, the internal cavity and the inlet conduits is transparent or substantially transparent (for example by being formed from materials such as nylon or polycarbonate) with the other components of the connector (e.g., the rotatable component, etc.) received within the housing formed from a material that is opaque (for example acetyl or nylon). This makes it possible for the components received within the housing to be visualised through the transparent housing so the operator has a visual check to see that component parts are properly aligned and close connections are formed at the junctions between the different components.

As noted above, a first portion of the internal cavity includes the first passageway. A second portion of the internal cavity that is adjacent to the first portion of the internal cavity is typically adapted to receive the rotatable component. Preferably the longitudinal axis of the first passageway is coaxial with a longitudinal axis of the second portion of the internal cavity that is adapted to receive the rotatable component and with the central axis of the housing (and hence also the rotational axis of the rotatable component received in the second portion of the internal cavity of the housing). However, in an alternative embodiment of the disclosure, the first passageway is offset from the central axis of the housing. Preferably the first passageway is formed in the internal cavity where a cross-sectional profile of the internal cavity narrows. In an embodiment of the disclosure, the first passageway terminates in an outlet port and preferably the outlet port is connectable to a catheter. In a further embodiment of the disclosure, the outlet port is an extension of the housing and may be integrally formed with it. Preferably an axis of the outlet port is coaxial with the rotational axis of the rotatable component (and hence may also be coaxial with the axis of the second portion of the internal cavity that is adapted to receive the rotatable component).

In an embodiment of the disclosure the first passageway terminates in a male or female Luer connector, commonly, a male Luer connector. The Luer connector formed at the end of the first passageway is connectable to a catheter or other conduit for conveying a fluid or suspension of particles towards the target in the subject's body.

In a further embodiment of the disclosure the first passageway comprises a circular or oval cross-sectional profile. Having a circular or oval cross-sectional profile reduces fluid turbulence within the first passageway (as compared to other cross-sectional profiles) and so has improved clearance of particles where the fluid being conveyed is a suspension of particles as particles cannot get trapped as easily. Where the cross-sectional profile of the first passageway is circular or oval in cross-sectional profile, it is desirable that the cross-sectional profile, including the dimensions, of the first end of the second passageway is the same. For example, where the first passageway has a circular cross-sectional profile, the first end of the second passageway should also have a circular cross-sectional profile of the same dimensions. Having matching cross-sectional profiles reduces turbulence at the junction of adjacent component parts caused by mechanically mismatched parts, and this improves the clearance of particles where the fluid being conveyed is a suspension of particles.

Inlet Conduits

Two or more inlet conduits are formed in the housing and are arranged radially with respect to the longitudinal axis of the first passageway. Preferably the longitudinal axis of the first passageway is coaxial with the rotational axis of the rotatable component and so in this case the two or more inlet conduits are also arranged radially with respect to the rotational axis of the rotatable component. Preferably the inlet conduits and the housing are integrally formed in a single moulded piece. Two or more (e.g., two, three, four, five, six, etc.) inlet conduits are formed in the housing. In certain beneficial embodiments of the disclosure the connector comprises four inlet conduits.

The two or more inlet conduits are connectable to feed lines for supplying fluid and/or a suspension of particles to the connector. Having two or more inlet conduits allows two or more feed lines to be connected to the connector and these feed lines are in turn connected to two or more vials (or other suitable containers) containing fluids. During a therapeutic procedure to administer radioactive microparticles in suspension, it is sometimes necessary to deliver the contents of more than one vial of radioactive microparticles. The present disclosure permits more than one vial to be connected to the system, and the operator can easily switch to the second vial once the contents of the first vial have been administered. It is also sometimes desirable to deliver non-therapeutic fluids in a procedure as well as a suspension of therapeutic particles, for example, a contrast agent in solution for visualising the procedure, and it is advantageous to be able to connect to two or more sources of fluid via two or more feed lines and to be able to switch between those sources of fluid easily, without having to disconnect and reconnect feed lines to the sources of fluid.

In an embodiment of the disclosure, at least one of the inlet conduits terminate in a male or female Luer connector compatible with a corresponding Luer connector on a feed line. For example, the inlet conduits may terminate in a female Luer connector compatible with a male Luer connector on a feed line.

In an embodiment of the disclosure, the inlet conduits are arranged such that they have a rotational symmetry with respect to the longitudinal axis of the first passageway. For example, in the embodiment of the disclosure where the connector comprises four inlet conduits the inlet conduits are arranged 90 degrees apart from each other with respect to the longitudinal axis of the first passageway such that they have a rotational symmetry of four. In an alternative embodiment of the disclosure where the connector comprises three inlet conduits, the inlet conduits are arranged 120 degrees apart from each other with respect to the longitudinal axis of the first passageway such that they have a rotational symmetry of three; in an alternative embodiment of the disclosure where the connector comprises five inlet conduits, the inlet conduits are arranged 72 degrees apart from each other with respect to the longitudinal axis of the first passageway such that they have a rotational symmetry of five; in an alternative embodiment of the disclosure where the connector comprises six inlet conduits, the inlet conduits are arranged 60 degrees apart from each other with respect to the longitudinal axis of the first passageway such that they have a rotational symmetry of six; and so forth.

In an embodiment of the disclosure, one or more of the inlet conduits may comprise a circular or oval cross-sectional profile. Having a circular or oval cross-sectional profile reduces fluid turbulence within the inlet conduits (as compared to other cross-sectional profiles) and so has improved clearance of particles as particles cannot get trapped as easily. Where the cross-sectional profiles of any one or more than one of the inlet conduits are circular or oval in cross-sectional profile, it is desirable that the cross-sectional profile, including the dimensions, of the second passageway (particularly the second passageway at the second end, which selectively alignable with any one of the inlet conduits) is the same. For example, where the second passageway has a circular cross-sectional profile, at least one of the inlet conduits will also have a circular cross-sectional profile of the same dimensions. Preferably all the inlet conduits will have a cross-sectional profile, including the dimensions, which are the same as the second end of the second passageway. Having matching cross-sectional profiles reduces turbulence at the junction of adjacent component parts caused by mechanically mismatched parts and this improves the clearance of particles where the fluid being conveyed is a suspension of particles.

Rotatable Component/Second Passageway

The rotatable component is received in the second portion of the internal cavity of the housing and is rotatable about the longitudinal axis of the second portion of the internal cavity and is also preferably rotatable about the longitudinal axis of the first passageway. As previously noted, the rotatable component comprises a second passageway having a first end in fluid communication with the first passageway, wherein the rotatable component is rotatable such that a second end of the second passageway is selectively alignable with any one of the inlet conduits such that a continuous flow path is established between the first passageway and the selected inlet conduit.

As also previously noted, the rotatable component is receivably engaged within a second portion of the internal cavity of the housing. Preferably the connector comprises a water-tight seal at the interface between the housing and the outer surface of the rotatable component. Preferably the housing has a smooth internal cavity, the second portion of which has a profile that is complimentary to the rotatable component. In some embodiments, the second portion of the internal cavity is cylindrical and the rotatable component has a cylindrical profile. In some embodiments, the second portion of the internal cavity is tapered (e.g., in the form of a truncated cone, corresponding to a cone without an apex) and is complementary to a taper (e.g., a complementary truncated cone) of the rotatable component. In an embodiment of the disclosure, the rotatable component is engaged within the internal cavity of the housing with a clip fit.

The rotatable component may be manually operated by hand, which is preferably achieved by turning a top part of the rotatable component which extends beyond the housing. In one embodiment of the disclosure, the top part of the rotatable component has a protruding section configured to be gripped by a human hand. Alternatively, in another embodiment of the disclosure, the top part of the rotatable component has a circular profile with ribs for gripping by the human hand.

By turning the rotatable component, the operator can select which of the inlet conduits to place in fluid communication with the second passageway and hence also the first passageway. The rotatable component may be able to turn within the housing between 180 degrees and 360 degrees (i.e. a full turn) with respect to the longitudinal axis of the first passageway, however this depends on the number inlet conduits provided on the connector. For example, if the connector has two inlet conduits preferably these will be arranged to be 180 degrees apart from each other with respect to the longitudinal axis of the first passageway and so the rotatable component will need to be able to rotate through a minimum of 180 degrees with respect to the longitudinal axis of the first passageway. In another example the connector has three inlet conduits arranged 120 degrees apart from each other with respect to the longitudinal axis of the first passageway and so the rotatable component will need to be rotatable through a minimum of 240 degrees with respect to the longitudinal axis of the first passageway to be able to select each of the inlet conduits. In still another example the connector has four inlet conduits arranged 90 degrees apart from each other with respect to the longitudinal axis of the first passageway and so the rotatable component will need to be rotatable through a minimum of 270 degrees with respect to the longitudinal axis of the first passageway to be able to select each of the inlet conduits. In an embodiment of the disclosure, the rotatable component is configured to indicate which of the inlet conduits is selected such that a continuous flow path is established between said inlet conduit and the first passageway.

The connector comprises two or more inlet conduits connectable to two or more sources of fluid and so the rotatable component can be used to select which of the sources of fluid, when connected to their respective inlet conduits, is selected to be in fluid communication with the first passageway. For example, when the connector comprises four inlet conduits, up to four separate sources of fluid may be connected to the connector at the same time and the rotatable rotatable component can be used to select which of the four sources of fluid is in fluid communication with the first fluid passageway. In preferred embodiments. the rotatable component comprises only one second passageway, and it is only possible for one of the two or more inlet conduits to be in fluid communication with the first passageway at a time. It is therefore only possible for fluid to flow from one source of fluid through the connector to the first passageway at any given time. So, in the example where four separate sources of fluid are connected to the connector, only one of them is able to be in fluid communication with the first passageway at any given time and the other three are closed off. In a further example, the connector comprises four fluid inlets with four sources of fluid connected to the connector, these being three vials containing a suspension of radioactive microparticles and one vial of contrast agent in solution. In this example the operator is able to operate the rotatable component to switch between administering radioactive microparticles from each vial in turn and alternating this with periodically administering contrast agent in solution from a separate vial to visualise the procedure.

Preferably the material or materials which make up the rotatable component are opaque, for example acetyl or nylon, and the housing is formed from a material that is transparent or substantially transparent such that the rotatable component can be visualised through the housing. This makes it possible for the rotatable component to be visualised through the transparent housing so the operator has a visual check to see that the second end of the second passageway is correctly aligned with the selected inlet conduit and that close connections are formed at the junctions between these components.

The second passageway is in fluid communication with the first passageway at a first end, and the second end of the second passageway is selectively alignable with one of the two or more inlet conduits. The angle between each of the inlet conduits and the first passageway is greater than 90 degrees and as explained above, this angle may be accommodated by a bend formed in the second passageway. As the bend is formed in the second passageway, rather than directly at a junction between a fluid inlet and the first passageway, this reduces the number of particles that become trapped between mechanically mismatched components because the bend is not formed at the junction between two components.

In an embodiment of the disclosure, the second passageway comprises a circular or oval cross-sectional profile. Having a circular or oval cross-sectional profile reduces fluid turbulence within the second passageway, as compared to other cross-sectional profiles, and so has improved clearance of particles as particles cannot get trapped as easily. In general, it is desirable that the cross-sectional profile, including the dimensions, of the first passageway be the same as that of the second passageway, at least where the first passageway interfaces with the second passageway. It is also desirable that the cross-sectional profile, including the dimensions, of the inlet conduits be the same as that of the second passageway, at least where the inlet conduits interface with the second passageway upon alignment. For example, where the second passageway has a circular cross-sectional profile, the first passageway typically has a circular cross-sectional profile of the same dimensions, at least where the first and second passageways interface with one another. Similarly, where the second passageway has a circular cross-sectional profile, the inlet conduits typically have a circular cross-sectional profile of the same dimensions, at least at the point where the second passageway and each of the inlet conduits are selectively aligned and interface with one another. This reduces to a minimum the existence of mechanically mismatched components at the junction between the first passageway and the second passageway and/or any one of the inlet conduits. This in turn reduces to a minimum the incidence of particles becoming trapped in gaps that exist between mechanically mismatched components. Preferably all the inlet conduits will have a cross-sectional profile and dimension which are the same as that of the second end of the second passageway, and the first passageway will also have a cross-sectional profile and dimension which is the same as that of the first end of the second passageway. It is also an advantage to have a consistent inner diameter throughout the flow path, from the inlet conduits through the second passageway and first passageway. This reduces the number of residual particles that get trapped because fluid turbulence is minimised by removing regions of stagnant fluid and flow eddys where the inner diameter widens. It also ensures that fluid flowing through the connector moves at a constant velocity.

System for Conveying a Suspension of Particles

In a further aspect of the disclosure, there is provided a system for conveying a suspension of particles that comprises (a) a first source of fluid, a second source of fluid and optionally one or more additional sources of fluid wherein at least one source of fluid is a suspension of particles, (b) a connector according to an aspect of the disclosure, (c) two or more feed lines for supplying fluid to the connector wherein each feed line is connected to an inlet conduit at a first end and to a source of fluid at a second end, and (d) a catheter in fluid communication with the first passageway.

System-Sources of Fluid

The system comprises a first source of fluid, a second source of fluid and optionally one or more additional sources of fluid wherein at least one source of fluid is a suspension of particles. A suspension of particles may for example be a suspension of embolic particles or a suspension of radioactive microparticles. Other sources of fluid can include contrast agent in solution for visualising a therapeutic procedure. Typically, a source of fluid is provided in a vial or any kind of container suitable for holding fluids. The sources of fluid are connectable to a feed line. Sources of fluid may be provided with a suitable connector, such as a male or female Luer coupling, among others.

System-Feed Lines

The system further comprises two or more feed lines for supplying fluid to the connector wherein each feed line is connected to an inlet conduit at a first end and to a source of fluid at a second end. Each end of each feed line may be provided with a suitable connector, such as a male or female Luer connector, among others.

System-Catheter

The system may further comprise a catheter in fluid communication with the first passageway. The catheter is connectable to an outlet formed for the first passageway, for example the catheter may connectable to the outlet via a male or female Luer connector.

System-Bracket

In an embodiment of this aspect of the disclosure, the system may further comprise a bracket configured for holding the connector in an orientation such that the clearance of particles through the connector towards the outlet (and thus also the catheter) is maximised. Preferably the bracket should be used such that the connector is oriented such that the longitudinal axis of the first passageway is aligned with a gravitational force field (e.g., straight up and down, which can be s measured, for example, by a plumb line). This optimises clearance of particles through the connector where the source of fluid is a suspension of particles. Preferably, the bracket should be rigid in order to prevent the catheter and/or feed lines from becoming twisted. The bracket may include, for example, a base, a vertical support (e.g., one that is perpendicular to the base), and a bracket arm (e.g., one that is perpendicular to the vertical support and parallel to the base).

Method of Conveying a Suspension of Particles

In another aspect of the disclosure, there is provided a method of conveying a suspension of particles through a system, comprising providing a system according to an aspect of the disclosure, using the rotatable component to select a first inlet conduit thereby delivering fluid through the system from a first source of fluid, rotating the rotatable component to select a second inlet conduit thereby delivering fluid through the system from a second source of fluid and optionally rotating the rotatable component to select a third inlet conduit thereby delivering fluid through the system from a third source of fluid. Optionally the rotatable component may be rotated to select fourth or further inlet conduits thereby delivering fluid through the system from a fourth or further sources of fluids. Preferably at least one of the sources of fluid will include a suspension of particles.

As such, a system according to the disclosure can be used to deliver fluid from two or more sources of fluid, sequentially one after the other or switching between the two or more sources as required. In one embodiment of the disclosure, the system may be used for the administration of a therapeutic agent and in this instance, it may also be desirable to administer contrast agent in solution in addition to the therapeutic agent to allow the procedure to be visualised. In this case one of the sources of fluid will be a vial containing contrast agent in solution and one or more additional vials will contain therapeutic agent. For example, the operator may start by administering an amount of contrast agent in solution from a first vial attached to the first inlet conduit in order to visualise the vasculature ahead of administering the therapeutic agent. The operator may then use the rotatable component to select the second inlet conduit which is attached to a second vial containing a suspension of therapeutic particles to administer them to the subject in question. The operator may then use the rotatable component to select the third inlet conduit which is attached to a third vial containing a suspension of therapeutic particles to administer an additional dose. Alternatively, the operator may re-select the first inlet conduit attached to the first vial containing contrast agent in solution to visualise the vasculature again before administering further therapeutic particles from the third vial. It is not possible to deliver contrast agent in solution at the same time as therapeutic particles during such a procedure but the operator may often find it desirable to be able to administer a small amount of contrast agent in solution pre-procedure and then again between each vial of therapeutic agent being administered to confirm that the therapeutic agent continues to reach its target.

### BRIEF DESCRIPTION OF THE DRAWINGS

FIG. 1 represents a cross-sectional view of the connector according to an aspect of the disclosure.

FIG. 2 represents an expanded partial view of the central portion of the connector with the angle between an inlet conduit and an axis of the first passageway shown and also the angle between an inlet conduit and the rotational axis of the rotatable component shown.

FIG. 3 represents a top view of the connector according to an aspect of the disclosure.

FIG. 4 represents a perspective view of the connector according to an aspect of the disclosure.

FIG. 5 shows a schematic representation of the system according to an aspect of the disclosure.

FIG. 6 represents a perspective view of a bracket which is configured for holding a connector according to an aspect of the disclosure.

FIG. 7 is a photograph of a bracket supported by a bracket arm according to an aspect of the disclosure.

FIG. 8 is a photograph of a connector positioned in a bracket supported by a bracket arm according to an aspect of the disclosure.

### DETAILED DESCRIPTION

A detailed description of one embodiment of the present disclosure will now be described with reference to the drawings.

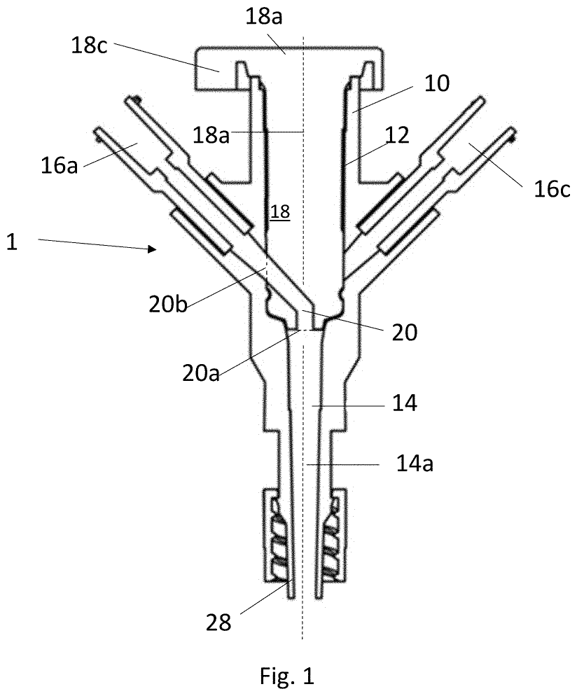

FIG. 1 represents a cross-sectional view of the connector (1) according to an aspect of the disclosure. In this embodiment of the disclosure, the longitudinal axis (14a) of the first passageway (14) is coaxial with the rotational axis (18a) of the rotatable component (18). The connector (1) comprises housing (10) comprising an internal cavity (12) that includes the first passageway (14) arranged about the longitudinal axis. Inlet conduits (16a, 16c) are formed in the housing (10) and arranged radially with respect to the longitudinal axis of the housing (10), which is coaxial with the passageway axis (14a) and the rotational axis (18a). In the cross-sectional view of the connector (1) shown in FIG. 1, only two of the inlet conduits (16a, 16c) can be seen out of four total inlet conduits (16a, 16b, 16c, 16d). The rotatable component (18) is received in a portion of the internal cavity (12) of the housing at a position adjacent to the first passageway (12), and rotatable component (18) is rotatable about its longitudinal axis (18a) which is coaxial with the axis (14a) of the first passageway (14). The rotatable component (18) comprises a second passageway (20) in fluid communication with the first passageway (14) at its first end (20a). The rotatable component is rotatable such that the second end (20b) of the second passageway is selectively alignable with any one of the inlet conduits (16a, 16b, 16c, 16d). In the embodiment of the disclosure shown in FIG. 1, the second end (20b) of the second passageway (20) is aligned with the first inlet conduit (16a) such that a continuous flow path is established between the first passageway (14) and the first inlet conduit (16a) (the first inlet conduit (16a) being the selected inlet conduit in this instance). In the embodiment of the disclosure shown in FIG. 1, the rotatable component (18) has a top part (18a) with a pointer (18c) which indicates that the first inlet conduit (16a) is the selected inlet conduit. In the embodiment of the disclosure shown in FIG. 1, the first passageway (14) terminates in an outlet port (28).

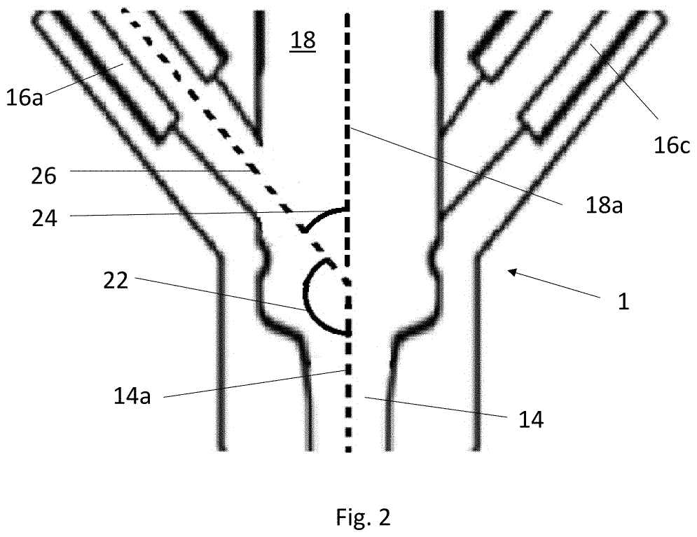

FIG. 2 represents an expanded view of the central portion of the connector (1) with the angle (22) between the axis (26) of the first inlet conduit (16a) and the axis (14a) of the first passageway (14) being shown and also the angle (24) between the axis (26) of the first inlet conduit and the rotational axis (18a) of the rotatable component (18) being shown. The central portion of the connector shown in FIG. 2 is a schematic representation, and the second passageway is not shown. In the embodiment of the disclosure shown in FIG. 2, the longitudinal axis (14a) of the first passageway (14) is coaxial with the rotational axis (18a) of the rotatable component (18) and where this is the case the sum of angle (22) and angle (24) should always equal 180 degrees. The angle (22) between the axis of the first inlet conduit and the axis of the first passageway should always be greater than 90 degrees.

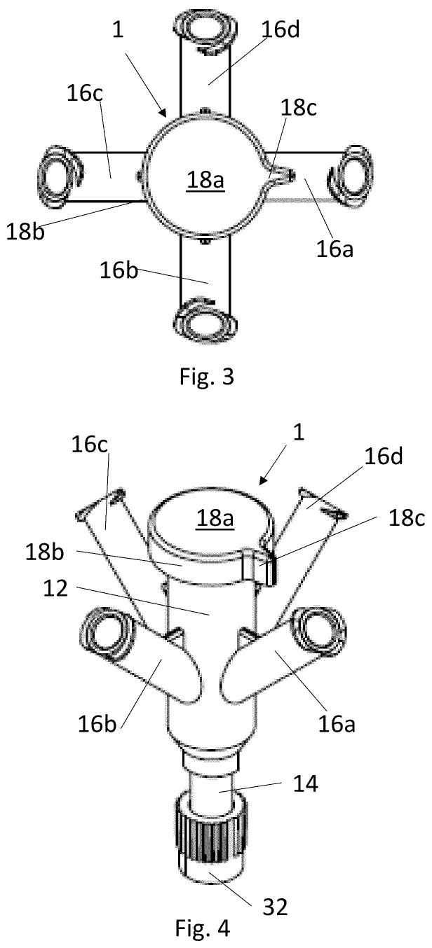

FIG. 3 represents a top view of the connector (1) and FIG. 4 represents a perspective view of the connector (1) according to an aspect of the disclosure. In the embodiment of the disclosure shown in FIGS. 3 and 4, the connector (1) has four inlet conduits (16a, 16b, 16c, 16d) and these inlet conduits are arranged 90 degrees apart from each other with respect to the longitudinal axis of the first passageway (14) such that they have a rotational symmetry of four. In the embodiment of the disclosure shown in FIGS. 3 and 4, the rotatable component has a top part (18a) which extends beyond the housing and also a protruding section (18b) configured to be gripped by a human hand. In the embodiment of the disclosure shown in FIGS. 3 and 4, the rotatable component also has a pointer (18c) for indicating which of the inlet conduits (16a, 16b, 16c, 16d) has been selected to be in fluid communication with the first passageway (14) such that a continuous flow path is established between the selected inlet conduit and the first passageway (14) via the second passageway (20). In the embodiment of the disclosure shown in FIGS. 3 and 4, the pointer (18c) is pointing towards the first inlet conduit (16a) to indicate that the first inlet conduit (16a) is the selected inlet conduit and so is in fluid communication with the first passageway (14). In the embodiment of the disclosure shown in FIG. 3, the first passageway (14) terminates in an outlet port (32).

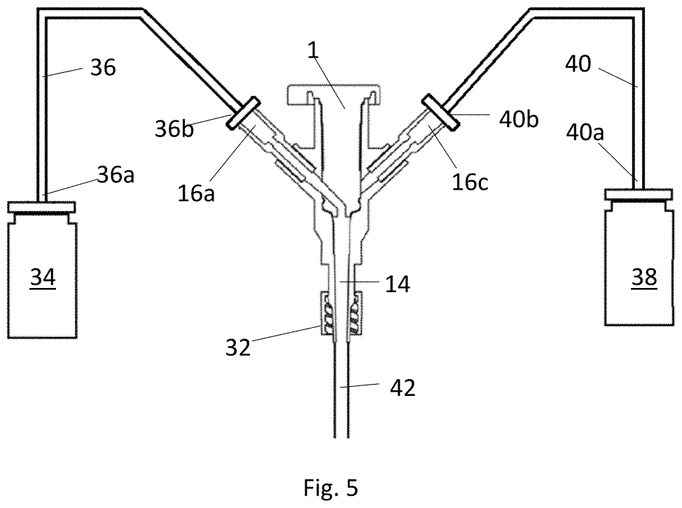

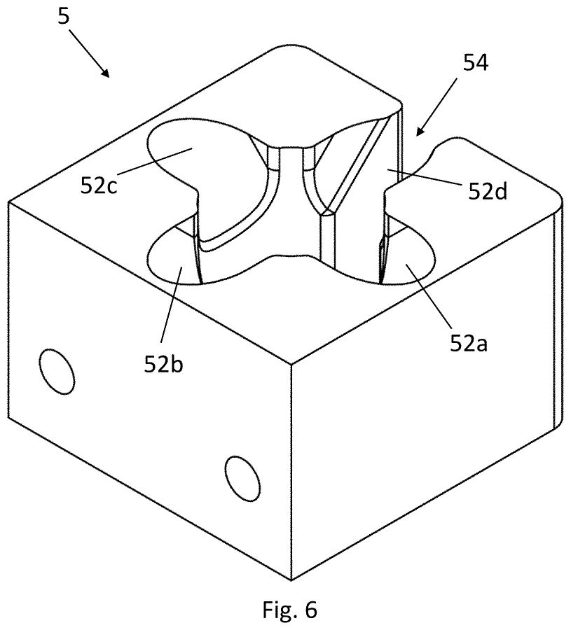

FIG. 5 shows a schematic representation of a system according to an aspect of the disclosure. The system comprises a connector (1) like that previously described, a first source of fluid (34) and a first feed line (36), wherein the first feed line (36) is connected to the first source of fluid (34) at a first end (36a) and to a first inlet conduit (16a) on the connector at its second end (36b). The system further comprises a second source of fluid (38) and a second feed line (40), wherein the second feed line (40) is connected to the second source of fluid (38) at a first end (40a) and to a second inlet conduit (16c) on the connector at a second end (40b). The system also comprises a catheter (42) in fluid communication with the first passageway (14) on the connector. In the embodiment of the disclosure shown in FIG. 5, the catheter (42) is attached to the first passageway (14) at its outlet port (32) via a Luer connector. In the embodiment of an aspect of the disclosure shown in FIG. 5, the system comprises two sources of fluid and two feed lines however optionally the system may also comprise one or more additional sources of fluid and a corresponding number of additional feed lines for connecting the additional sources of fluid to one or more additional inlet conduits on the connector. In the embodiment of the disclosure shown in FIG. 5, the first source of fluid and second sources of fluid (34, 38) are vials.

FIG. 6 represents a perspective view of a bracket (5) in accordance with an aspect of the present disclosure, which is configured for holding a connector (1) in accordance with an aspect of the present disclosure like that described above. The bracket (5) is comprises with four concavities (52a, 52b, 52c, 52d), each of which is configured to cradle each of four inlet conduits (16a, 16b, 16c, 16d). The bracket (5) is further provided with a slot (54) to allow the connector (1) to be seated in the bracket (5) and/or removed from the bracket (5) while a catheter (42) is attached to the connector (1).

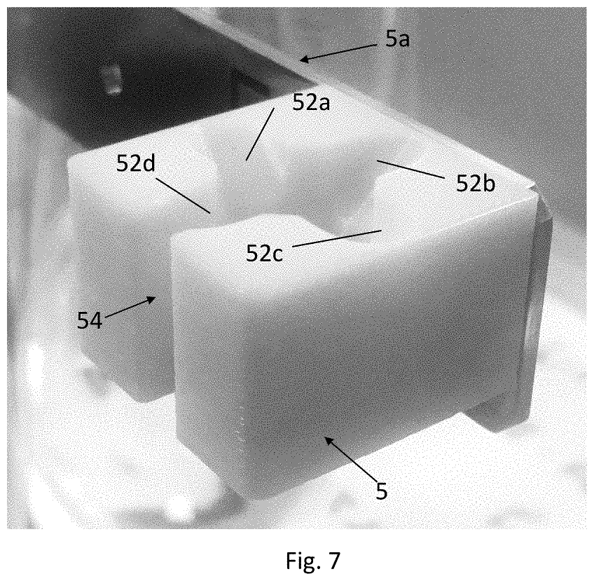

FIG. 7 is a photograph of a bracket (5) like that of FIG. 6 supported by a bracket arm (5a), in accordance with an aspect of the present disclosure.

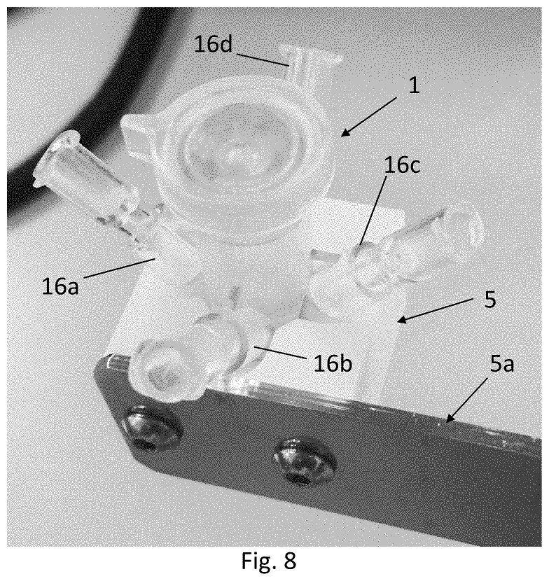

FIG. 8 is a photograph of a connector (1), in accordance with an aspect of the present disclosure, positioned in a bracket (5) supported by a bracket arm (5a) in accordance with an aspect of the present disclosure.

1. A connector for conveying a suspension of particles through a system, said connector comprising:
a housing (10) comprising an internal cavity (12) that comprises a first passageway (14) having a longitudinal axis for conveying fluid and wherein the first passageway terminates in an outlet;
two or more inlet conduits (16a, 16b, 16c, 16d) formed in the housing and arranged radially with respect to the longitudinal axis of the first passageway; and
a rotatable component (18) received in a portion of the internal cavity that is adjacent to the first passageway, the rotatable component being rotatable about the longitudinal axis of the first passageway, wherein the rotatable component comprises an internal second passageway (20) in fluid communication with the first passageway at a first end (20a) of the second passageway, and wherein the rotatable component is rotatable such that a second end (20b) of the second passageway is selectively alignable with any one of the inlet conduits such that a continuous flow path is established between the first passageway and a selected inlet conduit;
wherein an angle between a longitudinal axis of each of the inlet conduits and the longitudinal axis of the first passageway is greater than 90 degrees.

2. The connector according to claim 1, wherein the angle between the longitudinal axis of each of the inlet conduits and the longitudinal axis of the first passageway is greater than 110 degrees.

3. The connector according to claim 1, wherein the rotatable component is configured to indicate which of the inlet conduits is selected as the selected inlet conduit such that a continuous flow path is established through the second passageway between said selected inlet conduit and the first passageway.

4. The connector according to claim 1, wherein the first passageway terminates in an outlet port.

5. The connector according to claim 1, wherein the rotatable component is engaged within the internal cavity of the housing by a clip fit.

6. The connector according to claim 1, wherein the inlet conduits are arranged such that they have rotational symmetry with respect to the longitudinal axis of the first passageway.

7. The connector according to claim 1, wherein the connector comprises two, three or four inlet conduits.

8. The connector according to claim 1, wherein the first passageway and/or any one or more than one of the inlet conduits terminate in a Luer connector.

9. The connector according to claim 1, wherein the first passageway terminates in a male Luer connector and the one or more inlet conduits terminate in female Luer connectors.

10. The connector according to claim 1, wherein the first passageway, the second passageway, and/or any of one or more of the inlet conduits comprise a circular or oval cross-sectional profile.

11. The connector according to claim 1, wherein the housing is integrally formed in a single moulded piece.

12. The connector according to claim 1, wherein the longitudinal axis of the first passageway is coaxial with a central axis of the housing.

13. A system for conveying a suspension of particles, comprising:
two or more sources of fluid, wherein at least one source of fluid is a suspension of particles;
a connector comprising: a housing (10) comprising an internal cavity (12) that comprises a first passageway (14) having a longitudinal axis for conveying fluid and wherein the first passageway terminates in an outlet; two or more inlet conduits (16a, 16b, 16c, 16d) formed in the housing and arranged radially with respect to the longitudinal axis of the first passageway; and a rotatable component (18) received in a portion of the internal cavity that is adjacent to the first passageway, the rotatable component being rotatable about the longitudinal axis of the first passageway, wherein the rotatable component comprises an internal second passageway (20) in fluid communication with the first passageway at a first end (20a) of the second passageway, and wherein the rotatable component is rotatable such that a second end (20b) of the second passageway is selectively alignable with any one of the inlet conduits such that a continuous flow path is established between the first passageway and a selected inlet conduit; wherein an angle between a longitudinal axis of each of the inlet conduits and the longitudinal axis of the first passageway is greater than 90 degrees;
two or more feed lines for supplying fluid to the connector wherein each of the two or more feed lines is connected to each of the two or more inlet conduits at a first feed line end and each of the two or more feed lines is connected to each of the two or more sources of fluid at a second feed line end; and
a catheter in fluid communication with the first passageway.

14. A system according to claim 13, wherein the system further comprises a bracket configured for holding the connector in an orientation such that a clearance of particles through the connector towards the outlet is maximised.

15. The system according to claim 13, wherein the angle between the longitudinal axis of each of the inlet conduits and the longitudinal axis of the first passageway is greater than 110 degrees.

16. The system according to claim 13, wherein the rotatable component is configured to indicate which of the inlet conduits is selected as the selected inlet conduit such that a continuous flow path is established through the second passageway between said selected inlet conduit and the first passageway.

17. The system according to claim 13, wherein the inlet conduits are arranged such that they have rotational symmetry with respect to the longitudinal axis of the first passageway.

18. The system according to claim 13, wherein the connector comprises two, three or four inlet conduits and the system comprises two, three or four feed lines.

19. A method of conveying a suspension of particles through a system comprising (a) two or more sources of fluid, wherein at least one source of fluid is a suspension of particles; (b) a connector comprising: a housing (10) comprising an internal cavity (12) that comprises a first passageway (14) having a longitudinal axis for conveying fluid and wherein the first passageway terminates in an outlet; two or more inlet conduits (16a, 16b, 16c, 16d) formed in the housing and arranged radially with respect to the longitudinal axis of the first passageway; and a rotatable component (18) received in a portion of the internal cavity that is adjacent to the first passageway, the rotatable component being rotatable about the longitudinal axis of the first passageway, wherein the rotatable component comprises an internal second passageway (20) in fluid communication with the first passageway at a first end (20a) of the second passageway, and wherein the rotatable component is rotatable such that a second end (20b) of the second passageway is selectively alignable with any one of the inlet conduits such that a continuous flow path is established between the first passageway and a selected inlet conduit; wherein an angle between a longitudinal axis of each of the inlet conduits and the longitudinal axis of the first passageway is greater than 90 degrees; (c) two or more feed lines for supplying fluid to the connector wherein each of the two or more feed lines is connected to each of the two or more inlet conduits at a first feed line end and each of the two or more feed lines is connected to each of the two or more sources of fluid at a second feed line end; and (d) a catheter in fluid communication with the first passageway, the method comprising:
using the rotatable component to select a first inlet conduit of the two or more inlet conduits thereby delivering fluid through the system from a first source of fluid of the two or more sources of fluid to the catheter; and
rotating the rotatable component to select a second inlet conduit of the two or more inlet conduits thereby delivering fluid through the system from a second source of fluid of the two or more sources of fluid to the catheter.

20. The method according to claim 19, further comprising rotating the rotatable component to select a third inlet conduit of the two or more inlet conduits thereby delivering fluid through the system from a third source of fluid of the two or more sources of fluid to the catheter.
